(19) United States
(12) Patent Application Publication
(10) Pub. No.: US 2002/0020408 A1
(43) Pub. Date: Feb. 21, 2002

(54) Inhalation medicament delivery device

(75) Inventor: Peter M. Knauer, Sandy, UT (US)

Correspondence Address: BEYER WEAVER & THOMAS LLP, P.O. BOX 778, BERKELEY, CA, 94704-0778, US

(73) Assignee: Invivotech, Inc.

(21) Appl. No.: 09/865,961

(22) Filed: May 25, 2001

Related U.S. Application Data
(60) Provisional application No. 60/207,894, filed on May 25, 2000.

(51) Int. Cl. A61M 11/00

(52) U.S. Cl. 128/200.14; 128/200.23

(57) ABSTRACT

An inhalation medicament delivery device used to administer single or multiple dose of medicament. A standard vial container attaches to the device eliminating the need for a specialized vial container. The device is configured so that a dose can be accurately measured and transferred from the vial container to the device for delivery to the user. The device utilizes a vibration element attached to a membrane with fine holes to create particle sizes suitable for deep lung deposition. The device is breath activated, triggered only on intake of air, thereby reducing the loss of drug while exhaling. An electronic module tracks doses and provides historical data capable of being downloaded to a PC or PDA.

### FIELD OF THE INVENTION

This invention relates to medicament delivery devices. More specifically, the invention relates to self-administered inhalation medicament delivery devices.

### BACKGROUND OF THE INVENTION

Self administered subcutaneous or intramuscular injections are well known in the medical arts and have long been considered standard treatment, such as diabetics, who require fixed or measured doses of medicament on a regular and relatively frequent basis. Further, newer treatments, such as growth hormone injections, also require self administered injections on a regular basis.

Inhaled medicament delivery devices considerably reduce the burden to self administer medicament because they eliminate the use of needles. By eliminating needles, patients become more likely to administer the medicament on a regular basis, which increases its effectiveness. Furthermore, the cost per unit to deliver the medicament is reduced, because there is no need to replace a needle after every administration.

Typically, self administered injections are rendered using pre-filled syringes or cartridges of pre-measured doses of medicament. However, such self administered injections suffer from several drawbacks and are particularly ill suited for use by children or patients with disabilities because they required the patient to manually insert an exposed needle in the tissue.

Drug manufacturers spend significant cost and time producing pre-filled syringes or cartridges for self administered injection devices. As an alternative, a traditional vial, with a glass body and rubber stopper, is the least expensive and most readily available medicament container system. The vial is however, the least convenient system for the patient to use because an empty syringe must be inserted into the vial to draw out a medicament dose prior to an injection.

Prior art inhalation medicament delivery devices utilize specialized medicament containers that are even more costly to produce than pre-filled syringes or cartridges. These containers can not take advantage of the economies of scale since they are used in a relatively small amount of delivery systems. Furthermore, the materials used for these containers are typically a material not used previously with that medicament, and require a lengthy series of studies to determine whether the medicament degrades over time during storage.

Prior art inhalation medicament delivery devices consist of five major components: (i) a housing formed to contain electro-mechanical parts; (ii) a pre-filled container holding the medicament; (iii) a mechanical dosing mechanism that receives the medicament container; (iv) an electronic particle generating and controller assembly and (v) an aspiration tube to deliver the medicament into the lungs. Each of the five major components are described in detail below.

Prior art housing assemblies are used to hold the container in place during and after the delivery process. The housing joins the medicament container to the electro-mechanical particle generating system. The housing sometimes also holds a liquid crystal display to provide feedback to the patient during the delivery process.

Prior art medicament containers consist of a polymer container that holds either a dry powder of aqueous solution. The container has either a septum port or membrane with fine holes to transfer the medicament into the device.

Prior art mechanical dosing mechanisms receive the medicament container and enable the transfer of the medicament into the device in preparation for particle generation. The mechanism also measures the amount of dose to be delivered.

Prior art electronic particle system generates particles via a vibration system. Medicament is forces through a vibrating membrane with fine, uniform holes. The resulting particles are of a highly uniform nature, which facilitates deep lung deposition and high rates of absorbance.

Prior art aspiration tubes deliver the particles through the mouth and into the lungs. The tube mixes air with the particles in a precise manner and controls particle velocity in such a way that the majority of particles are delivered deep into the lungs.

### SUMMARY OF THE INVENTION

In the summary, the present invention is an inhalation medicament delivery device and corresponding method for use with a disposable membrane assembly having a membrane and a vial container that contains a medicament. The device comprises an aspiration tube, a disposable transfer system, a dosing mechanism, a control mechanism, and a housing. The disposable transfer system is configured to enable a user to attach thereto the vial container and the disposable membrane assembly. The dosing mechanism is configured to enable the user to attach thereto the disposable transfer system with the vial container and disposable membrane assembly attached. The housing configured to house the aspiration tube, the disposable transfer system, the dosing mechanism, and the particle generation mechanism. The housing is also configured to enable the user to attach to the dosing mechanism the disposable transfer system with the vial container and the disposable membrane assembly. The control mechanism is configured to enable the user to cause the dosing mechanism and the disposable transfer system to cooperatively measure and transfer a dose of the medicament from the vial container into the disposable transfer system after the disposable transfer system and the disposable membrane assembly are attached to the dosing mechanism. The control mechanism is further configured to, when the user breathes in on the aspiration tube to create air flow in the aspiration tube, cause the membrane to vibrate and cause the disposable transfer system to pressure the medicament of the dose against the membrane such that particles of the medicament are formed and drawn by the air flow through the aspiration tube and into the lungs of the user.

### BRIEF DESCRIPTION OF THE DRAWINGS

FIG. 1 is a cut view drawing of an inhalation medicament delivery device in accordance with the present invention.

FIG. 2 is an isometric view drawing of the inhalation medicament delivery device of FIG. 1.

FIG. 3 is cut view drawing of the disposable transfer system, standard vial container and disposable membrane assembly.

FIG. 4 is cut view drawing of the electronic particle generating system of the inhalation medicament delivery device of FIGS. 1 to 2.

FIG. 5 is a cut view drawing highlighting measuring a dose of medicament and transferring the dose into the disposable transfer system of the inhalation medicament delivery device of FIGS. 1 to 2.

FIG. 6 is a cut view drawing highlighting administering a dose of medicament of the inhalation medicament delivery device of FIGS. 1 to 2.

FIG. 7 is a cut view drawing highlighting the feed back system of the inhalation medicament delivery device of FIGS. 1 to 2.

### DETAILED DESCRIPTION

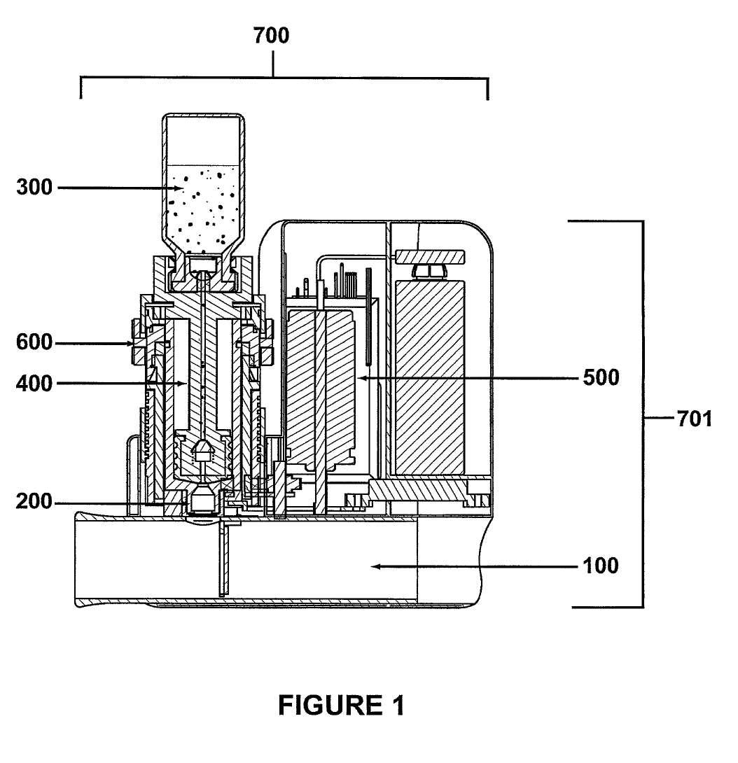

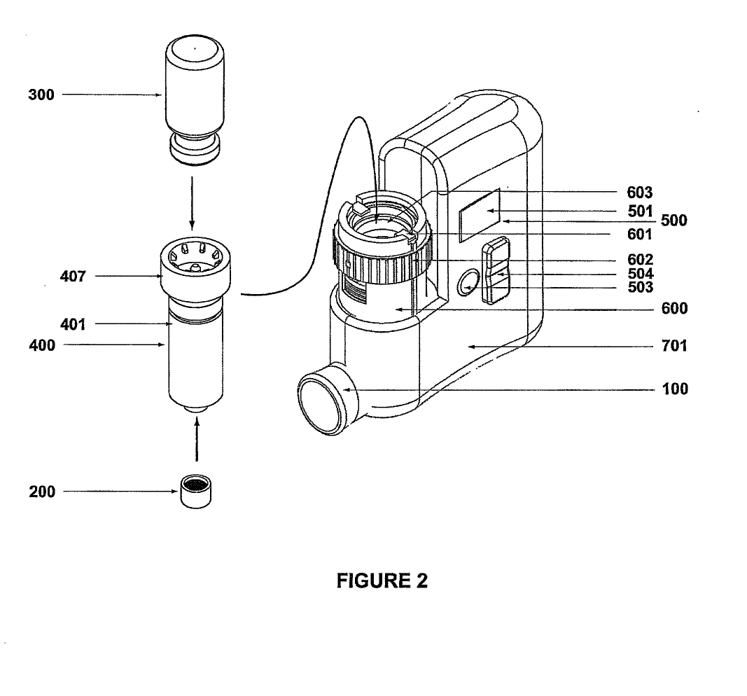

Referring to FIGS. 1 and 2, there is shown an inhalation medicament delivery device [700] according to one configuration (or embodiment) of the invention. The device comprises a housing (or body) [701] that houses an aspiration tube [100], a mechanical dosing mechanism [600], a control mechanism [500], and a disposable transfer system [400]. The device is used in conjunction with a standard glass vial container [300] containing a medicament and a disposable membrane assembly [200]

To provide the proper context for describing the inhalation medicament delivery device [700] in more detail later, a brief general description of the configuration and operation of the disposable transfer system [400] and the mechanical dosing mechanism [600] will be provided here. First, the standard glass vial container [300] is irreversibly attached to the disposable transfer system by snapping the standard glass vial container into place. This eliminates the need for a specialized vial container for the device [700]. The disposable membrane assembly [200] is removably attached to the front end of the disposable transfer system. The disposable transfer system, with the standard glass vial container and disposable membrane assembly attached, is inserted into and removably attached to the mechanical dosing mechanism [600] in preparation for injection. A measured (or set) dose of the medicament is transferred from the standard glass vial container into the disposable transfer system by selectively setting a dose via the dose toggle button [504] of the control mechanism [500]. The user (e.g., patient or caregiver) initiates the medicament administration by pressing a trigger button [503] of the control mechanism and breathing in on the aspiration tube [100]. The flow of air in the direction of the lungs triggers the control mechanism [500] to cause fine, uniform particles to be generated. The particles flow into the aspiration tube at an even rate and into the lungs for deposition. If the user breathes out, the resulting reversal of air flow turns the electronic particle generator off until flow is returned towards the direction of the lungs. The user continues this cycle until the set dose of medicament is completely used up, whereby the electronic particle generator automatically turns off.

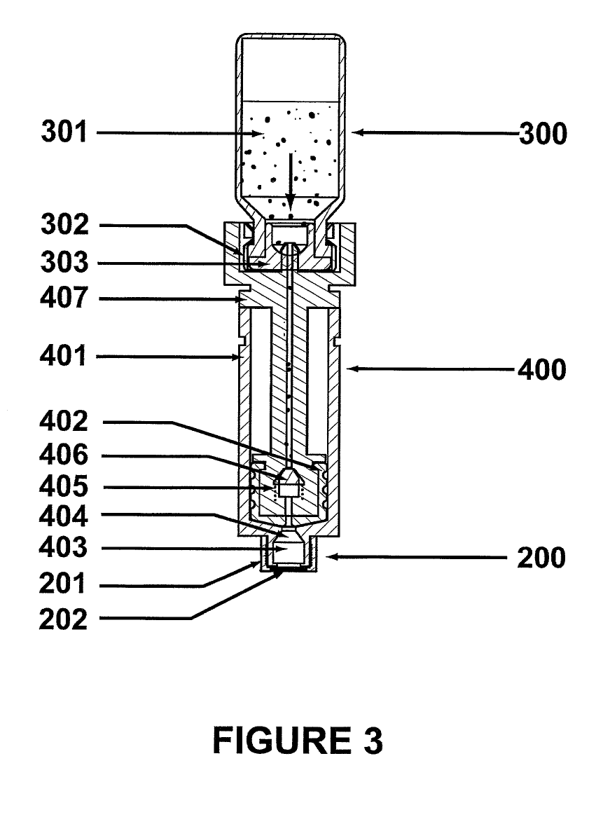

FIG. 3 shows some of the component parts of the standard vial container [301 to 303], the disposable transfer system [401 to 407], and the disposable membrane assembly [201 to 202]. These component parts will be discussed in conjunction with the more detailed discussion of the configuration and operation of the disposable transfer system [400] and the mechanical dose mechanism [600].

FIG. 3 also shows the details of attaching the disposable transfer system [400] to the mechanical dose mechanism [600] so that the disposable transfer system and the mechanical dose mechanism can work cooperatively in measuring (or setting) and delivering a dose of the medicament in the glass vial container [100]. The glass vial container is first snapped onto the plunger rod [407] of the disposable transfer system. Clips on the plunger rod are designed to bend away from the cap [302] of the standard glass vial container. Once the cap passes by, the clips grip the neck of the glass vial [301], irreversibly locking and snapping it into place. A hollow spike on the plunger rod penetrates the rubber stopper [303] of the standard glass vial container as the standard glass vial container is snapped into place. This opens a pathway for the medicament to pass from the standard glass vial container, through the plunger rod [407] and rubber plunger [402], and into the syringe barrel [401]. Once the standard vial container is attached, the disposable membrane assembly [200] is reversibly attached by threading the housing [201] onto the threads of the syringe barrel [401].

Referring to FIGS. 2 and 3, the disposable transfer system [400], with the standard glass vial container [300] and disposable membrane [200] attached, is then attached to the mechanical dose mechanism [600] by aligning the grooves on the plunger rod [407] and syringe barrel [401] with the tabs on upper lock ring [601] and lower lock ring [603]. The disposable transfer system can be reversibly removed (to be exchanged with a new one after use) by twisting the release ring [602] counter clockwise to pull the tabs away from the slots in the plunger rod and syringe barrel.

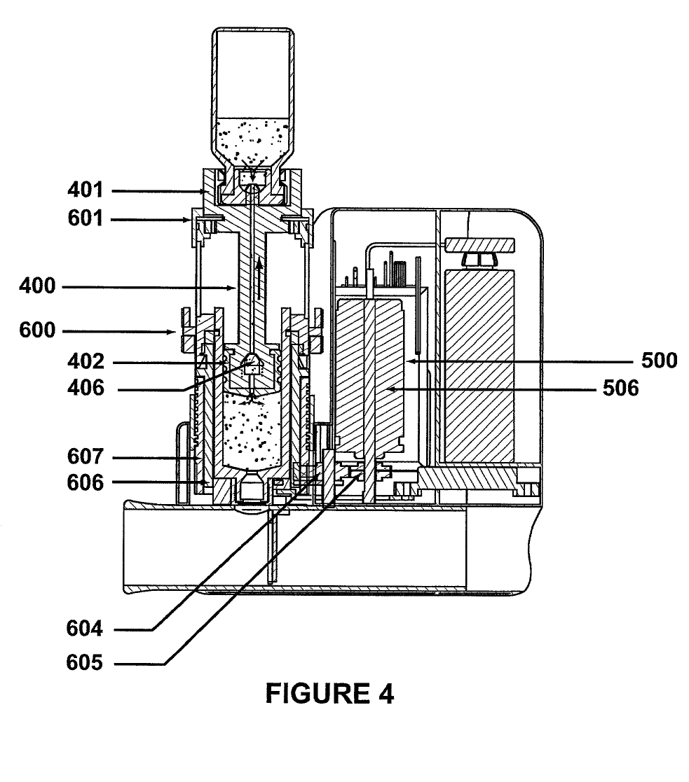

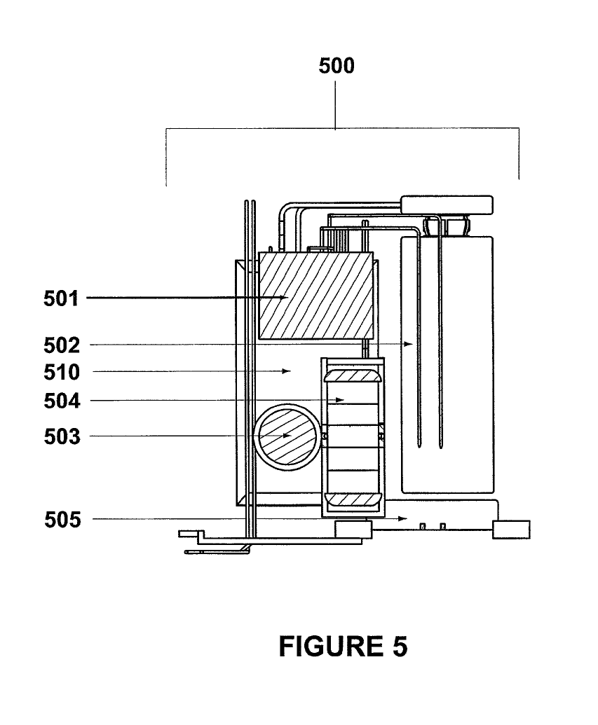

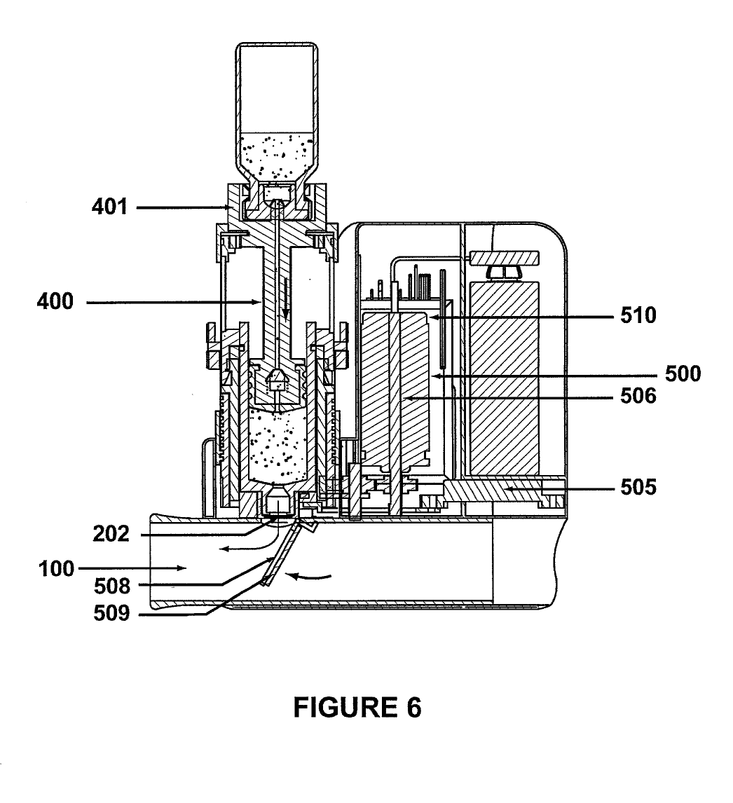

FIG. 4 shows the details of measuring (or setting) and transferring a dose into the disposable transfer system [400] using the mechanical dose mechanism [600]. Once the plunger rod [401] is locked into the upper lock ring [601], the user can measure (or set) a dose of medicament into the disposable transfer system by pressing the dose toggle [504, FIGS. 2 and 5] until a desired amount is registered on the liquid crystal display [501, FIGS. 2 and 5] of the control mechanism [500]. The electronic module [510, FIG. 5] of the control mechanism then turns the stepper motor [506, FIG. 6] of the control mechanism a corresponding amount of rotation, which turns the dose barrel a prescribed amount via drive gears 1 and 2 [604 and 605]. The rotation of the dose barrel [606] causes the translation barrel [607] to translate via interlocking threads on the dose barrel and translation barrel. The precise amount of rotation from the dose barrel results in a precise translation of the translation barrel, and its attached parts; the plunger rod [407], rubber plunger [402], check valve 1 [406], and standard vial container [300]. The corresponding translation of the plunger rod and rubber plunger transfers the medicament from the vial into the syringe barrel [401] in preparation for dose delivery. As the plunger rod translates away from the syringe barrel [401], a volume is created between the rubber plunger and the syringe barrel. The seal between the rubber plunger and syringe barrel ensures that the volume is created under a vacuum. The medicament in the vial, under ambient pressure, is pushed into the volume between the rubber plunger and syringe barrel. Check valve 1 [406] prevents the flow of medicament back into the vial when the direction of the rubber plunger is reversed during dose delivery. Check valve 2 seals the volume between the rubber plunger and syringe barrel.

FIG. 5 shows the details of some of the components [501 to 505 and 510] of the control mechanism [500]. The electronic module [510] includes a display, such as an LCD (liquid crystal display) [501], to display the measured dose from the electronic data. The electronic module is capable of tracking the measured dose history from administration to administration and is capable of downloading the electronic data representing the measured dose history to a remote personal computer or other device. The dose toggle button [504] is used to enter the amount of medicament to be transferred into the disposable transfer assembly prior to administration. The amount entered corresponds to the amount of rotation and translation of the mechanical dose mechanism. Trigger button [503] is used when the user is ready to administer the dose, as described in detail below. A standard 9 volt battery [502] provides power for the electronic module. Vibration for the particle generating system is provided by a peizo-vibration element [505] that may be piezoelectric or ultrasonic

FIG. 6 shows the process of delivering the medicament to the lungs. The measured dose is administered when the user presses the trigger button [503, FIGS. 2 and 5] and breathes in on the aspiration tube [100]. The flow of air through the aspiration tube [100] towards the lungs causes flap valve 1 [508] of the control mechanism [500] to swing open, sending a signal triggering the electronic module [510] to reverse the stepper motor [506] rotation, reversing the translation of the plunger rod [401] and rubber plunger [402]. The volume between the rubber plunger and syringe barrel [401] contracts and pressures the medicament against the membrane [202] of the disposable membrane assembly [200, FIG. 1]. The electronic module simultaneously causes the vibration element [505] to vibrate the membrane. The medicament is forced through the array of fine holes in the membrane by the pressure and the vibration of the vibration element. Uniform particles are generated as the columns of medicament passing through the membrane are broken off by the vibrational frequency of vibration element. Particles emerging from the membrane are drawn into the air flow of the aspiration tube in into the lungs. The proper combination of particle size (via membrane hole diameter and vibrational frequency) and velocity (aspiration tube diameter) result in a high medicament payload suitable for deep lung deposition.

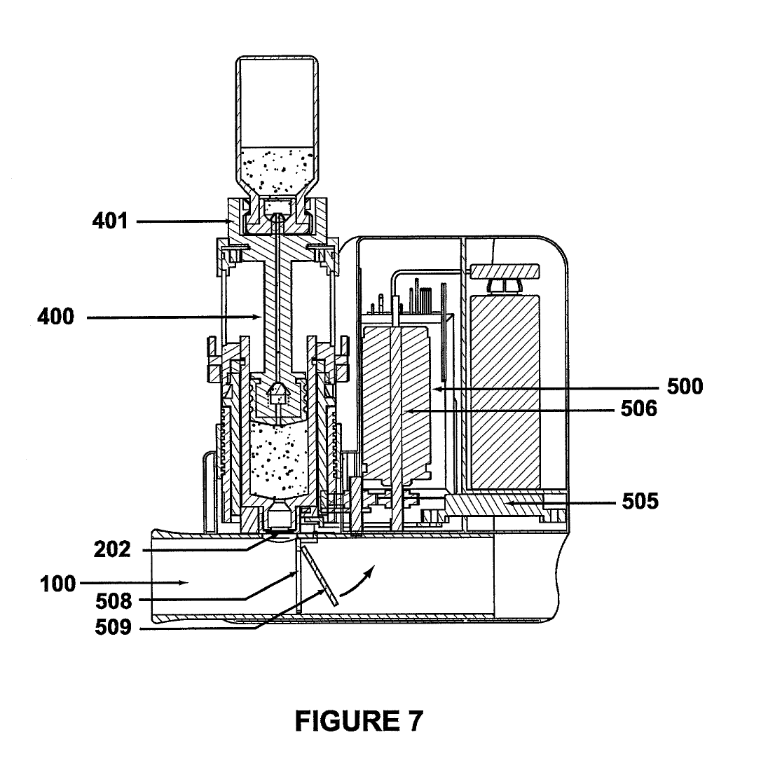

FIG. 7 shows details of the user feedback system. When the user breathes outwards, reversing the airflow in the aspiration tube [100], flap valve 1 [508] closes and send a signal to the electronic module to shut off the stepper motor [506] and vibration element [505], which stops the generation of particles. Flap valve 2 [509] of the control mechanism [500] opens in order to keep the user breathing in a normal pattern (prevents the aspiration tube from becoming blocked). This reduces the loss of the medicament while the user exhales. The next time the user breathes in, flap valve 1 opens again and flap valve 2 closes, returning the signal to the electronic module to start the stepper motor and vibration element to start generating particles once again. The user continues this cycle until all the medicament transferred into the disposable transfer assembly is gone, ending the administration cycle and automatically shutting down the inhalation medicament delivery device [700].

The aspiration tube [100] can be removably attached to the housing [701] with a bayonet pin and slot system or a friction fit. The inhalation medicament delivery device [700] can therefore be easily cleaned by removing the aspiration tube, washing it with soap and water, and then re-attaching it when clean.

As those skilled in the art will appreciate, a user can use the inhalation medicament delivery device [700] to repeatedly and accurately extract and measure doses of the medicament from the glass vial container [300] until it is empty. In doing so, the measured doses can be varied from dose to dose, as required by the user, or a fixed dose can be used every time.

A detailed description of the operation of one configuration of the invention is provided above. While this description of the invention is made with reference to a specific configuration, the description is only illustrative of the invention and is not to be construed as limiting the invention. Various modifications to the present invention can be made to the configuration described above by those skilled in the art without departing from the true spirit and scope of the invention as defined by the appended claims.

What is claimed is:

1. An inhalation medicament delivery device for use with a disposable membrane assembly having a membrane and a vial container that contains a medicament, the device comprising:
an aspiration tube;
a disposable transfer system configured to enable a user to attach thereto the vial container and the disposable membrane assembly;
a dosing mechanism configured to enable the user to attach thereto the disposable transfer system with the vial container and disposable membrane assembly attached;
a control mechanism; and
a housing configured to (a) house the aspiration tube, the disposable transfer system, the dosing mechanism, and the particle generation mechanism, and (b) enable the user to attach to the dosing mechanism the disposable transfer system with the vial container and the disposable membrane assembly;
the control mechanism being configured to (a) enable the user to cause the dosing mechanism and the disposable transfer system to cooperatively measure and transfer a dose of the medicament from the vial container into the disposable transfer system after the disposable transfer system and the disposable membrane assembly are attached to the dosing mechanism, and (b) when the user breathes in on the aspiration tube to create air flow in the aspiration tube, cause the membrane to vibrate and cause the disposable transfer system to pressure the medicament of the dose against the membrane such that particles of the medicament are formed and drawn by the air flow through the aspiration tube and into the lungs of the user.
